(19) United States
(12) Patent Application Publication
(10) Pub. No.: US 2025/0114479 A1
(43) Pub. Date: Apr. 10, 2025

(54) TISSUE-SELECTIVE SINGLE-COMPONENT MRNA DELIVERY CARRIER AND DELIVERY SYSTEM

(71) Applicant: INSTITUTE OF PROCESS ENGINEERING , CHINESE ACADEMY OF SCIENCES, Beijing (CN)

(72) Inventors: Xin ZHANG, Beijing (CN); Jun YANG, Beijing (CN); Zhiguo LU, Beijing (CN); Yiwan GENG, Beijing (CN)

(73) Assignee: INSTITUTE OF PROCESS ENGINEERING , CHINESE ACADEMY OF SCIENCES, Beijing (CN)

(21) Appl. No.: 18/981,653

(22) Filed: Dec. 16, 2024

Related U.S. Application Data
(63) Continuation of application No. PCT/CN2023/080186, filed on Mar. 8, 2023.

(51) Int. Cl. A61K 48/00

(52) U.S. Cl. CPC A61K 48/0041

(57) ABSTRACT

The present application is related to a tissue-selective single-component mRNA delivery carrier and a delivery system. Poly (β-amino ester) polymer is used as the single-component delivery carrier, wherein the poly (β-amino ester) polymer has a structure as shown in Formula I or Formula II. In Formula I or Formula II, the definition of each substituents is the same as in the detailed description. The present invention employs only the poly(β-amino ester) polymer material as the single delivery carrier, and tissue-selective targeting of the delivery system including lung, liver and spleen, etc. can be achieved by locally regulating the chemical structures and the domain charges of the poly(β-amino ester) polymer.

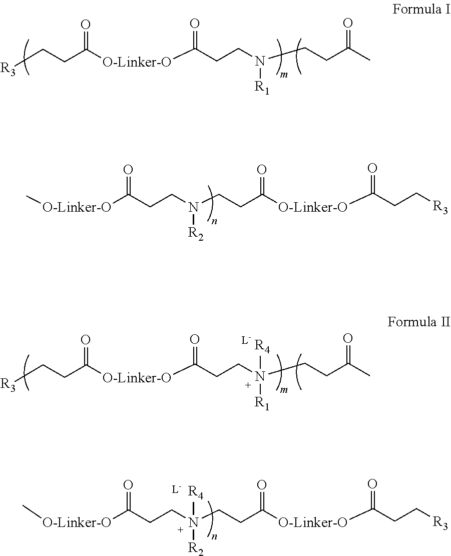

### CROSS-REFERENCE TO RELATED APPLICATION

The present application is a continuation of international application serial no. PCT/CN2023/080186 filed on Mar. 8, 2023. The entirety of the above-mentioned patent application is hereby incorporated by reference herein and made a part of this specification.

### BACKGROUND OF THE INVENTION

### Technical Field

The present invention pertains to the field of biomedical technology, and specifically focuses on a tissue-selective single-component mRNA delivery carrier and a delivery system.

### Description of Related Art

Since the successful expression of messenger RNA (mRNA) in vivo was first achieved in animal experiments in 1990, mRNA has been developed and applied to infectious disease prevention, tumor immunotherapy, protein replacement therapy, etc. mRNA can be utilized as vaccines or drugs to express target proteins in vivo; more importantly, compared with DNA, mRNA is translated in cells and does not enter the nucleus, so there is no risk of integration into the human genome.

mRNA is characterized by transient activity, high safety, fast production speed, and short research and development cycle, and it can be manipulated to obtain different target proteins and immune antigens for various diseases by changing the encoded mRNA. For example, the two new coronavirus disease 19 (COVID-19) mRNA vaccines approved for marketing in 2020 are vaccines which deliver the mRNA encoding the spike glycoprotein(S) into the body, and the synthesis of S protein in the body stimulates the production of the corresponding antibodies to achieve the purpose of prevention. In existing research and clinical trials, mRNA encoding tumor-associated proteins or immune-stimulating factors has been used in the treatment of various cancers, such as lung cancer, melanoma, and breast cancer.

Compared with other gene drugs, mRNA has an extra-large size, high electronegativity, and high sensitivity to enzymatic degradation in vivo, which make it difficult to be efficiently delivered from the injection site to the targeted cells for expression. Therefore, lipid nanoparticles (LNP) delivery system have been developed for mRNA delivery and have achieved certain success. Their delivery carriers mainly include: cationic or ionizable lipid, polyethylene glycol (PEG) lipid molecules, structural lipid molecules cholesterol, and auxiliary phospholipid molecules, which respectfully responsible for the functions of loading mRNA, protecting mRNA, maintaining the stability of the system, and assisting in the escape from lysosomes/endosomes,. However, PEG-containing drugs are prone to cause severe allergic reactions, and antibodies are easily produced during repeated administration that lead to the rapid clearance of the system; and the presence of multiple components makes it more difficult for drug formulations. More importantly, these lipid nanoparticles are prone to be enriched in liver, which makes it difficult to achieve the therapeutic needs of the diseases in extrahepatic tissues.

Herein, mRNA delivery systems that enable specific organ targeting have been reported. For example, CN113786391A discloses a method of preparation and application of a lung-targeted mRNA drug delivery system, wherein said mRNA delivery system comprising quaternary ammonium salt lipid molecules and lipid molecules for loading the mRNA, enabling the lung-targeted mRNA delivery with high specificity. However, these existing systems only target delivery in a single tissue (e.g., lung), making it difficult to achieve specific targeted delivery to various tissues. Also, the components of the delivery system are often more than one, making it difficult for drug formulation, and the toxic and side effects are diverse. It is difficult to achieve the treatment needs for multiple diseases with a single delivery system. Therefore, the design of a simple and efficient organ-specific targeted nucleic acid drug delivery system to achieve multi-diseases treatments is the research focus in the technical field.

### SUMMARY OF THE INVENTION

The purpose of the present invention is to provide a tissue-selective single-component mRNA delivery system, which only uses poly(β-amino ester) as the single delivery carrier, and achieves targeted enrichment of the system in various tissues in vivo, including lung, liver, spleen and so on by locally regulating the chemical structures and the microregion charge of poly(β-amino ester) polymers.

The technical solution provided by the present invention is:

A tissue-selective single-component mRNA delivery carrier, which uses the poly(β-amino ester) polymer as a single component delivery vector, and the poly(β-amino ester) polymer includes a structure represented by Formula I or Formula II:

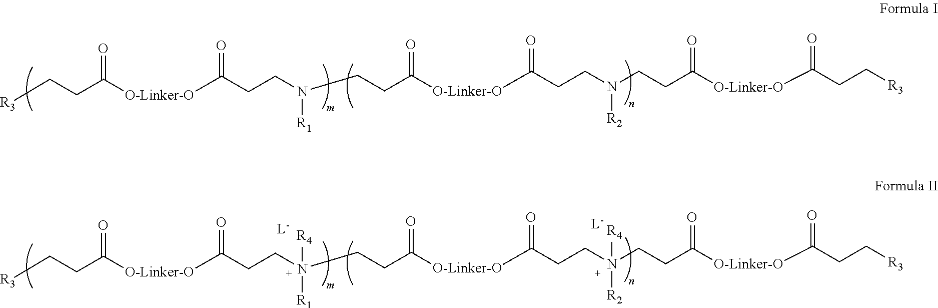

wherein,
Linker is selected from one of bisacrylate-containing compounds;
R₁ is selected from one of hydrophilic amine compounds;
R₂ is selected from one of hydrophobic amine compounds;
R₃ is selected from the groups of linear chain or branched chain of diamino-compounds;
R₄ is selected from halogenated hydrocarbons; and
L is a monovalent anion; and
m:n=1:1˜100, wherein m and n each independently represents the numbers of the polymerization of the poly (β-amino ester) polymer.

As a preferred embodiment, the molecular weight of the poly(β-amino ester) polymer range from 2000 Da to 20000 Da.

As a preferred embodiment, the bisacrylate-containing compounds include ethylene glycol diacrylate, bis(ethylene glycol) diacrylate, 1,3-butanediol diacrylate, 1,4-butanediol bisacrylate, 1,5-pentanediol diacrylate, 1,6-hexanediol diacrylate, ditriglycol diacrylate, tetraethylene glycol diacrylate, neopentyl glycol diacrylate, 1,10-decanediol diacrylate, 3-methyl-1,5-pentanediol diacrylate, ethoxylated bisphenol A diacrylate, bisphenol A glycerol diacrylate, polyethyleneglycol diacrylate, triethylene glycol diacrylate, poly (propyleneglycol) diacrylate, fluorescein O,O′-dipropyl diacrylate, and 2-propenoic acid-(2-hydroxy-1,3-propenylidene)bis[oxy(2-hydroxy-3,1-propylidene)] ester.

As a preferred embodiment, the hydrophilic amine compounds include N,N-dimethylethylenediamine, 3-dimethylamino-1-propylamine, 3-diethylaminopropylamine, N,N-diethylethylenediamine, ethanolamine, 4-amino-1-butanol, 2-amino-2-propanol, 2-amino-2methyl-1-propanol, 2-(2-amino)ethoxy)ethanol, N-(3-aminopropyl) morpholine, N-(2-aminoethyl)piperazine, 1-(3-aminopropyl)imidazole, 1-(3aminopropyl)-2-methyl-1-imidazole, 2-amino-2-methyl-1,3-propanediol, 2-amino-1,3-propanediol, 2-amino-5-diethylaminopentane, 1-(2-aminoethyl)piperidine, 3-piperidinylpropanamine, 2-fluoroethylamine, cysteamine, 2-(4-hydroxyphenyl)ethylamine, 4-aminotetrahydrofuran, (3-aminopropyl)trimethoxysilane, 3-aminopropyl(diethoxy)methylsilane, bradypropylamine, 3-pyridinylmethylamine, 5-hydroxydopamine, galactosamine, 1-methoxy-2-propylamine, 2-(ethylthio)ethylamine, 1-pyridinyl-ethylamine, 2-(2-pyridinyl)ethylamine, cysteamine S-phosphate, 6-amino-1-hexanethiol, 2-(2-methylimidazolyl)ethylamine, 2-(1-pyrrol-1-yl)ethylamine, tris(hydroxymethyl)aminomethane, 3-hydroxy-4-methoxybenzene ethanamine, aminoglycol dimethanol, and aminoglycol diethanol.

As a preferred embodiment, the hydrophobic amine compounds include n-hexylamine, n-heptylamine, n-octylamine, n-nonylamine, n-decylamine, n-dodecylamine, n-tridecylamine, n-tetradecylamine, n-hexadecylamine, n-octadecylamine, isoamylamine, iso-octylamine, 6-methyl-1-heptylamine, 2-nonylamine, 2-aminotridecylamine, 6-aminoundecane, 2-ethyl-1-hexylamine, 3-dibutylaminopropylamine, mercapturic undecylamine, oleylamine, and geranylamine.

As a preferred embodiment, the linear chain or branched chain of the diamino-compounds include ethylenediamine, 1,3-diaminopropane, 1,4-butanediamine, 1,5-pentanediamine, 1,6-hexanediamine, 1,7-diaminoheptane, 1,8-diaminooctane, 1,9-diaminononane, 1,10-diaminodecane, 1,11-undecanediamine, 1,12-dodecanediamine, 1,3-pentanediamine, 1,4-diamino cyclohexane, 1,3-diamino-2-propanol, cystamine, 2,2-bis(aminoethoxy)propane, p-phenylenediamine, 1,2-phenylenediamine, o-phenylenediamine, m-phenylenediamine, m-phenylenediamine, polyethylene glycol bis(amine), 2,2-dimethyl-1,3-propanediamine, 1,2-diaminopropane, 2,2′-oxo-bis(ethylammonium), 1,2-cyclohexanediamine 3,6,9-trioxaundecane-1,11-diamine, 4,9-dioxa-1,12-dodecanediamine, N,N-bis(3-aminopropyl)methanamine, 4,7,10-trioxa-1,13-tridecanediamine, 1,3-cyclohexanedimethanamine, and 4,4′-bisdiphenylphosphine methane(cyclohexylamine).

As a preferred embodiment, the halogenated hydrocarbons include iodomethane,

bromomethane, iodoethane, ethyl bromide, bromopropane, iodopropane, 2-iodopropane, 2-iodo-2-methylpropane, 1-iodo-2-methylpropane, isopropyl bromide, bromobutane, 1-bromo-3-methylbutane, 1-bromo-2-ethylbutane, 2-bromo-2-methylpropane, iodobutane, 2-iodobutane, and 1-iodo-3-methylbutane.

The preparation methods of the poly(β-amino ester) polymer include the following:

### I. Preparation of the Poly(β-Amino Ester) Polymer Represented by Formula I

(1) subjecting a bisacrylate Linker, a hydrophilic amine monomer R₁ and a hydrophobic amine monomer R₂ to a Michael addition polymerization reaction in a proportion;
(2) end-capping the polymer prepared in step (1) with a diamine monomer R₃ to obtain the poly (β-amino ester) polymer represented by Formula I.

### II. Preparation of the Poly(β-Amino Ester) Polymer Represented by Formula II

(1) subjecting a bisacrylate Linker, a hydrophilic amine monomer R₁ and a hydrophobic amine monomer R₂ to a Michael addition polymerization reaction in a proportion;
(2) end-capping the polymer prepared in step (1) with a diamine monomer R₃ to obtain the poly(β-amino ester) polymer represented by Formula I;
(3) reacting the poly(β-amino ester) polymer represented by Formula I prepared in step (2) with a halogenated hydrocarbon R₄, and converting a tertiary amine in the polymer into a quaternary amine to obtain the poly(β-amino ester) polymer represented by Formula II.

As a preferred embodiment, in step (1), the molar ratio between the bisacrylate Linker and the total amount of the hydrophilic amine monomer R₁ and the hydrophobic amine monomer R₂ is 1.01˜1.20:1.

As a preferred embodiment, in step (1), the reaction solvent for the Michael addition polymerization is selected from at least one of N,N-dimethylformamide, dimethyl sulfoxide, tetrahydrofuran, acetonitrile, dioxane, methanol, ethanol, isopropanol, or ethyl acetate; the reaction temperature ranges from 25-120° C., and the reaction time is from 2 h to 7 d.

As a preferred embodiment, in step (2), the molar ratio of diamine monomer R₃ and bisacrylate Linker is 0.01˜0.20:1.

As a preferred embodiment, in step (2), the solvent for the end-capping reaction is selected from at least one of N,N-dimethylformamide, dimethyl sulfoxide, tetrahydrofuran, acetonitrile, dioxane, methanol, ethanol, isopropanol, or ethyl acetate; the reaction temperature ranges from 25-120° C., and the reaction time is from 2 h to 7 d.

As a preferred embodiment, in step (3), the amount of halogenated hydrocarbon R₄ is 0.1-10 times the total molar amount of hydrophilic amine monomer R₁ and hydrophobic amine monomer R₂.

As a preferred embodiment,, in step (3), the reaction solvent is selected from at least one of N,N-dimethylformamide, dimethyl sulfoxide, tetrahydrofuran, acetonitrile, dioxane, methanol, ethanol, isopropanol, or ethyl acetate; the reaction temperature ranges from 25-40° C., and the reaction time is from 2 h to 72 h.

In any aspect, the tissue-selective mRNA delivery system comprises a delivery carrier and a mRNA loaded on the delivery carrier.

As a preferred embodiment, the preparation method for the delivery system is as follows: dissolving the poly (β-amino ester) polymer in an organic solvent to obtain an organic phase, and diluting the mRNA with an aqueous phase buffer to obtain an aqueous phase of the mRNA; and mixing the organic phase and the aqueous phase of the mRNA to obtain the mRNA delivery system.

As a preferred embodiment, the mRNA includes but is not limited to luciferase mRNA, enhanced green fluorescent protein mRNA, Cy5 fluorescently labeled mRNA, etc.

The mRNA delivery system provided in the present invention has the following advantages:

### (1) Single Component, Which is Beneficial for Drug Development

The previously reported nano-delivery carriers for mRNA mainly consist two or more components, with complex formulations, and numerous adjustment and evaluation of the components' proportion and efficacy are required. Whereas in the mRNA delivery system of the present invention, the poly(β-amino ester) polymer used as the delivery carrier has only a single component, which has a highly adjustable structure, and is easy for preparation, which greatly reduces the work of evaluating the performance of the proportions of each component, and the single-component carrier is very beneficial for subsequent drug development.

### (2) Multi-Organ Tissue Selective Targeting In Vivo

In the mRNA delivery system provided in the present invention, mRNA can be efficiently delivered to various tissues (such as lung, liver and spleen) in vivo through adjusting the chemical structures and changes in microregion charge of the poly(β-amino ester) polymer. Fine-tuning the structure of a single component can achieve the effect of selective targeted delivery to multiple organs. Expression of the mRNA delivery system in liver and spleen can be achieved by regulating the ratio of hydrophilic and hydrophobic side-chain in the polymers. By increasing the proportion of hydrophobic side-chain in the polymer, expression of the mRNA of the delivery system will be enriched in the spleen. By changing the degree of quaternization of the delivery carrier, expression of the mRNA of the delivery system in lung and spleen can be achieved. And by increasing the degree of quaternization of the delivery carrier, expression of the mRNA of the delivery system will be targeted to lung.

In any aspect, the mRNA delivery systems provided in the present invention can achieve the effect of tissue-selective targeting in vivo, such as lung, liver and spleen, and development of drug application in these tissues.

As a preferred embodiment, the organic solvent is at least one of alcohols, or dimethyl sulfoxide, and the pH of the aqueous buffer solution range from 4 to 6.5.

As a preferred embodiment, the concentration of the poly(β-amino ester) polymer in the organic solvent is 0.1˜10 mg/mL; and the mRNA concentration in aqueous solution is 5˜500 ng/μL.

As a preferred embodiment, the mass ratio of poly(β-amino ester) polymer to mRNA in the provided mRNA delivery systems is 1˜200:1.

As a preferred embodiment, the administration method of the mRNA delivery system is selected at least one of intravenous injection, intramuscular injection, intradermal injection, subcutaneous injection, intrathecal injection, or intraperitoneal injection.

The beneficial effects brought by the present invention are:

The mRNA delivery systems provided in the present invention utilizes poly(β-amino ester) polymer as the single component delivery carrier, which has a single component and is easy to prepare, And utilizes a single carrier to achieve the effect of targeted delivery to multiple tissues in vivo.

The chemical structures and charge degree of the delivery carrier in the present invention can be regulated, and multi-tissues targeting can be realized by adjusting the structures and microregion charge of the polymers. mRNA can be loaded on to the carriers efficiently and targeted delivery to different tissues in various organs can be achieved, making mRNA drugs have a very broad application in the treatments of multiple diseases.

### BRIEF DESCRIPTION OF THE DRAWINGS

FIG. 1 shows the nuclear magnetic spectrum of APO18-20 prepared in Example 1.

FIG. 2 shows the nuclear magnetic spectrum of APO18-8 prepared in Example 2.

FIG. 3 shows the nuclear magnetic spectrum of CNC18-10-2 prepared in Example 3.

FIG. 4 shows the expression distribution of the mRNA delivery system provided in Example 4 in mice (A), the average radiance (B) and the percentage of total radiance in organs (C). H: heart, L: lung, Li: liver, Sp: spleen, K: kidney.

FIG. 5 shows the expression distribution of the mRNA delivery system provided in Example 5 in mice (A), the average radiance (B) and the percentage of total radiance in organs (C). H: heart, L: lung, Li: liver, Sp: spleen, K: kidney.

FIG. 6 shows the expression distribution of the mRNA delivery system provided in Example 6 in mice (A), the average radiance (B) and the percentage of total radiance in organs (C). H: heart, L: lung, Li: liver, Sp: spleen, K: kidney.

FIG. 7 shows the expression distribution of the mRNA delivery system provided in Comparative Example 1 in mice (A), the average radiance (B) and the percentage of total radiance in organs (C). H: heart, L: lung, Li: liver, Sp: spleen, K: kidney.

FIG. 8 shows the expression distribution of the mRNA delivery system provided in Comparative Example 2 in mice (A), the average radiance (B) and the percentage of total radiance in organs (C). H: heart, L: lung, Li: liver, Sp: spleen, K: kidney.

### DETAILED DESCRIPTION OF THE EMBODIMENTS

The technical solutions are further explained by the detailed examples.

In any aspect, unless specified, the materials and equipment used in the present invention can be purchased commercially, or are commonly used in the technical field. The methods in the following embodiments, unless specified, are conventional methods in the technical field.

### Example 1: Synthesis of APO18-20

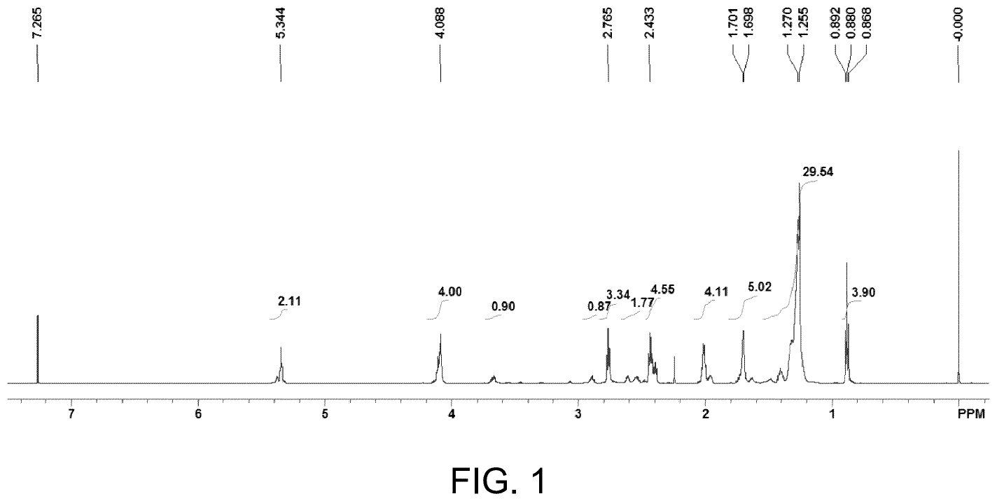

(1) 1,4-butanediol diacrylate (1.1 mmol), 2-amino-1,3-propanediol (0.0476 mmol) and oleylamine (0.952 mmol) are dissolved in appropriate amount of N,N-dimethylformamide in a 25 mL round-bottom flask. The mixture is stirred and heated to 80° C. for 48 h to obtain the poly(β-aminoester) polymer.
(2) 1,4-butanediamine (0.1 mmol) is dissolved in N,N-dimethylformamide, and added to the polymer solution prepared in step (1). The mixture is reacted at 80° C. for 24 h. After the reaction, the mixture is dialyzed for several days to remove the reaction solvent and unreacted small molecular compounds. Finally, the end-capped poly(β-aminoester) polymer APO18-20 (FIG. 1) is obtained with a yield of 78.2%.

### Example 2: Synthesis of APO18-8

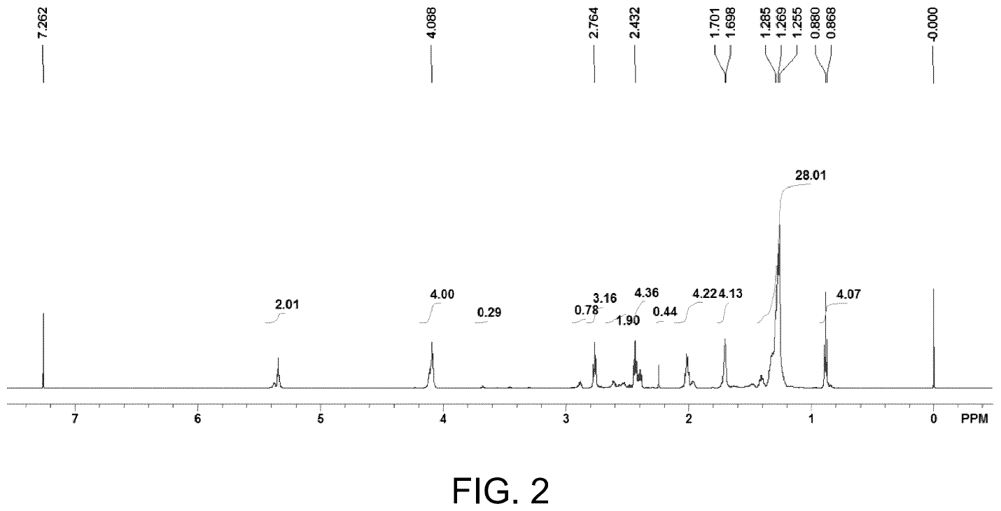

(1) 1,4-butanediol diacrylate (1.1 mmol), 2-amino-1,3-propanediol (0.11 mmol) and oleylamine (0.89 mmol) are dissolved in appropriate amount of N,N-dimethylformamide in a 25 mL round-bottom flask. The mixture is stirred and heated to 80° C. for 48 h to obtain the poly(β-aminoester) polymer.
(2) 1,4-butanediamine (0.1 mmol) is dissolved in N,N-dimethylformamide, and added to the polymer solution prepared in step (1). The mixture is reacted at 80° C. for 24 h. After the reaction, the mixture is dialyzed for several days to remove the reaction solvent and unreacted small molecular compounds. Finally, the end-capped poly(β-aminoester) polymer APO18-8 (FIG. 2) is obtained with a yield of 80.1%.

### Example 3: Synthesis of CNC18-10-2

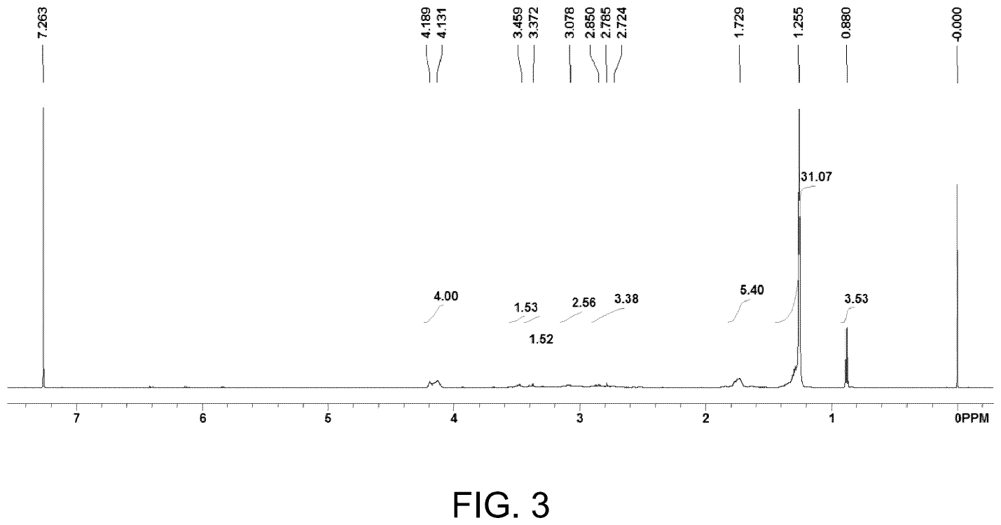

(1) 1,4-butanediol diacrylate (1.1 mmol), N,N-dimethylenediamine (0.091 mmol) and n-octadecylamine (0.91 mmol) are dissolved in appropriate amount of N,N-dimethylformamide in a 25 mL round-bottom flask. The mixture is stirred and heated to 80° C. for 48 h to obtain the poly (β-aminoester) polymer.
(2) 1,4-butanediamine (0.1 mmol) is dissolved in N,N-dimethylformamide, and added to the polymer solution prepared in step (1). The mixture is reacted at 80° C. for 24 h. After the reaction, the mixture is dialyzed for several days to remove the reaction solvent and unreacted small molecular compounds. Finally, the end-capped poly (β-aminoester) polymer ANC18-10 is obtained.
(3) The poly(β-aminoester) polymer ANC18-10 is dissolved in N,N-dimethylformamide, and iodomethane (4 mmol) is added dropwise. The mixture is stirred at 30° C. for 24 h, and then is dialyzed to remove the reaction solvent and unreacted small molecular compounds. Finally, the quaternized poly(β-aminoester) polymer CNC18-10-2 (FIG. 3) is obtained with a yield of 71.3%.

Similarly, various poly(β-aminoester) polymers can be synthesized by replacing 1,4-butanediol diacrylate with other diacrylate compounds, such as 6-hexanediol diacrylate, dipropylene glycol diacrylate, tetraethylene glycol diacrylate, neopentyl glycol diacrylate, 1,10- decanediol diacrylate, 3-methyl-1,5-pentanediol diacrylate, ethoxylated bisphenol A diacrylate, and so on, which will not be listed in detail here.

### Example 4

The preparation method of a mRNA delivery system, which comprises a delivery vector and a luciferase mRNA (commercially available) loaded on the vector, is listed below:
(1) obtaining the poly(β-aminoester) polymer APO18-20, and dissolving it in ethanol by ultrasound to prepare a stock solution with a concentration of 10 mg/mL;
(2) adding 5 μL of ethanol to 10 μL of the above-mentioned poly(β-aminoester) polymer APO18-20 stock solution in step (1) to obtain a final volume of 15 μL to make the organic phase;
(3) taking 2 μL from the 1 mg/mL luciferase mRNA stock solution, carefully adding sodium acetate buffer (pH 5.2) to mix, and resulting a final volume of 45 μL of the aqueous phase, containing 2 μg of mRNA;
(4) mixing the organic and aqueous phases from step (2) and (3) gently, homogenizing by vortexing, and diluting to 200 μL with PBS buffer pH 7.4 to obtain the mRNA delivery system.

### Examples 5-6

The preparation method of mRNA delivery systems is the same as those in Example 4, except that the poly(β-aminoester) polymer is changed to APO18-8 or CNC18-10-2.

### Comparative Example 1

(1) dissolving an appropriate amount of 1,2-dimyristoyl-rac-glycerin-3-methoxypolyethylene glycol 2000 (DMG-PEG2000), distearoyl phosphatidylcholine (DSPC), heptadecane-9-yl-8-((2-hydroxyethyl)6-oxo-6-(undecyl)hexyl) amino) octanoate (SM-102), and cholesterol (Chol) in ethanol by ultrasound to obtain the stock solutions with concentrations of 1 mg/mL, 1 mg/mL, 10 mg/mL and 10 mg/mL, respectively;
(2) mixing 2.43 μL of DMG-PEG2000, 5.09 μL of DSPC, 2.29 μL of SM-102, and 0.96 μL of Chol from step (1) and diluting with ethanol to obtain the final volume of the organic phase system of 15 μL, and the molar ratio of lipid molecules DMG-PEG2000:DSPC:SM-102:Chol in the organic phase is 1.5:10:50:38.5;
(3) taking 2 μL from the 1 mg/mL luciferase mRNA stock solution, carefully adding sodium citrate buffer (pH 4.0) to mix, and resulting a final volume of 45 μL of the aqueous phase, containing 2 μg of mRNA;
(4) mixing the organic phase in step (2) and aqueous phase in step (4) by using microfluidic method, with a flow rate of 150 μL/min for the aqueous phase and 50 μL/min for the organic phase, respectively. The mixed solution is further diluted with PBS buffer pH 7.4 to a final volume of 200 μL, and the Comparative Example 1 of mRNA delivery system is obtained.

### Comparative Example 2

(1) dissolving an appropriate amount of 1,2-dimyristoyl-rac-glycerin-3-methoxypolyethylene glycol 2000 (DMG-PEG2000), distearoyl phosphatidylcholine (DSPC), 4-(N,N-dimethylamino)butyric acid (6Z, 9Z, 28Z, 31Z)-heptadecacarbon-6,9,28,31-tetraen-19-yl ester (MC3) and cholesterol (Chol) in ethanol by ultrasound to obtain the stock solutions with concentrations of 1 mg/mL, 1 mg/mL, 10 mg/mL and 10 mg/mL, respectively;
(2) mixing 2.43 μL of DMG-PEG2000, 4.92 μL of DSPC, 2.00 μL of MC3, and 0.93 μL of Chol from step (1) and diluting with ethanol to obtain the final volume of the organic phase system of 15 μL, and the molar ratio of lipid molecules DMG-PEG2000:DSPC:MC3:Chol in the organic phase is 1.5:10:50:38.5;
(3) taking 2 μL from the 1 mg/mL luciferase mRNA stock solution, carefully adding sodium citrate buffer (pH 4.0) to mix, and resulting a final volume of 45 μL of the aqueous phase, containing 2 μg of mRNA;
(4) mixing the organic phase in step (2) and aqueous phase in step (4) by using microfluidic method, with a flow rate of 150 μL/min for the aqueous phase and 50 μL/min for the organic phase, respectively. The mixed solution is further diluted with PBS buffer pH 7.4 to a final volume of 200 μL, and the Comparative Example 2 of mRNA delivery system is obtained.

### Evaluation of the Targeting of mRNA Delivery System In Vivo

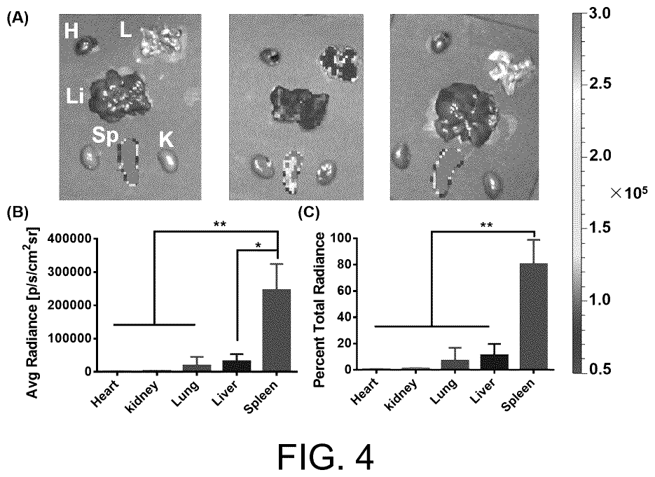

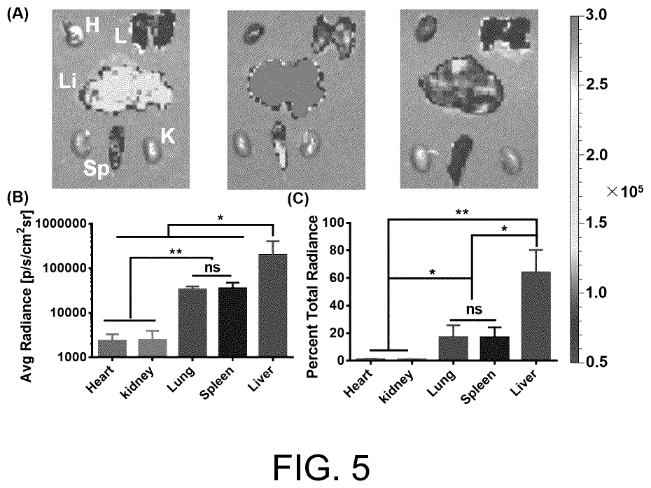

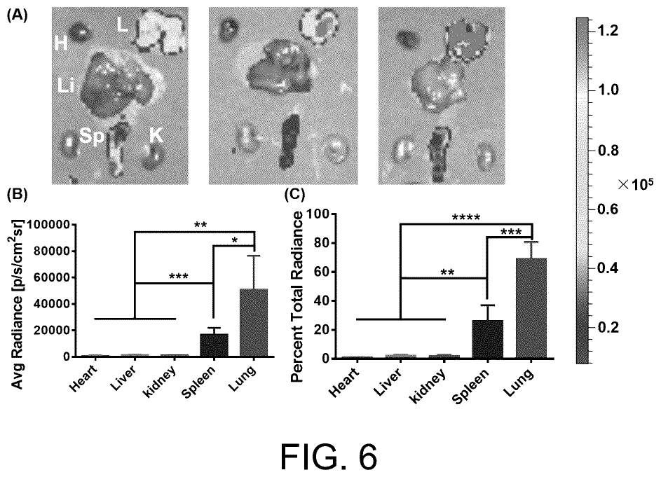

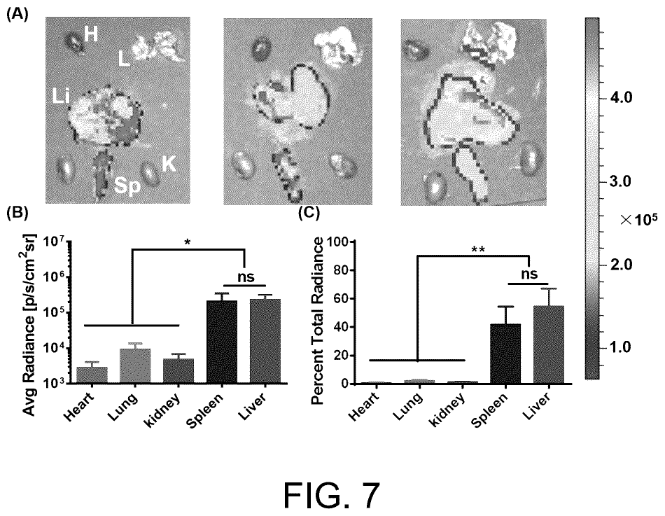

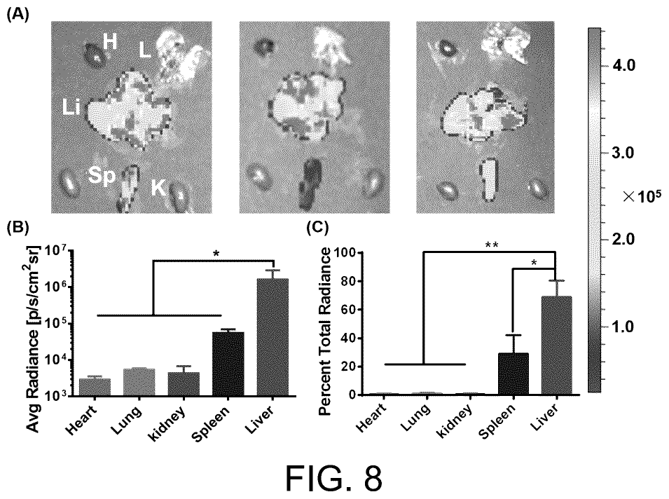

C57BL/6 mice is used as the experimental animals. The mRNA delivery systems provided in the present invention and the comparative examples are administrated through tail vein injection (the exemplary in vivo experimental results of Examples 4-6 and Comparative Examples 1-2 are shown). The administration dose for mRNA for each mouse is 0.1 mg/kg. After 6 h of administration, the luciferase substrate is injected via intraperitoneal administration at a dose of 150 mg/kg. After 10 minutes, the main organs of the mouse, including heart, liver, spleen, lung and kidneys, are removed for imaging to detect the protein expression of luciferase. FIGS. 4-6 demonstrate the distribution of luciferase protein expression in mice of Examples 4-6, while the FIGS. 7-8 exhibit the distribution of luciferase protein expression in mice of Comparative Examples 1-2. A quantitative analysis of photon flux per unit volume of each organs is performed (Avg Radiance [p/s/cm2sr]) by using the Living Image Software for bioluminescence. The percentage result graft is calculated by comparing the photon flux values per unit volume of each organ with the photon flux values of all organs (in Avg Radiance [p/s/cm2sr]). As the results shown, the protein expression of APO18-20 delivery system is mainly expressed in spleen tissue, accounting for 80.3%. The proportion of APO18-8 mRNA delivery system in liver tissue increased significantly compared to other tissues, reaching 64.0%. The CNC18-10-2 mRNA delivery system is mainly enriched in lung tissue, accounting for 69.0%. Under the same dosage of mRNA, the protein expression of Comparative Example 1 is mainly enriched in the liver and spleen, with the expression ratios of 54.5% and 41.6%, respectively. And the protein expression of Comparative Example 2 is mainly enriched in the liver, accounting for 68.8%. These results demonstrate that the mRNA delivery system in the present invention realizes the loading and delivery of mRNA with single component compared with lipid delivery systems which contain four components. More importantly, they can selectively target to various tissues by only adjusting the chemical structures and microregion charges of the poly(β-aminoester) polymers. These characteristics of the mRNA delivery system of the present invention facilitate druggability, and the multi-target delivery performance enable it to meet the treatment needs of multiple diseases in different tissues.

The applicants declare that the present invention illustrates the mRNA delivery systems, the preparation methods and applications of the mRNA delivery systems. However, the present invention is not limited to the above description, that is, it does not mean that the implementation of the present invention must rely on the above description. The technicians in the relevant technical field should understand that any improvement to this invention, equivalent substitution of the materials used in the invention, addition of auxiliary components, selection of the specific methods, and so on, all of these fall within the scope of protection and disclosure of the present invention.

What is claimed is:

1. A tissue-selective single-component mRNA delivery carrier, wherein the delivery carrier is a single delivery carrier formed by a poly(β-amino ester) polymer, the poly(β-amino ester) polymer has a structure represented by Formula I or Formula II: 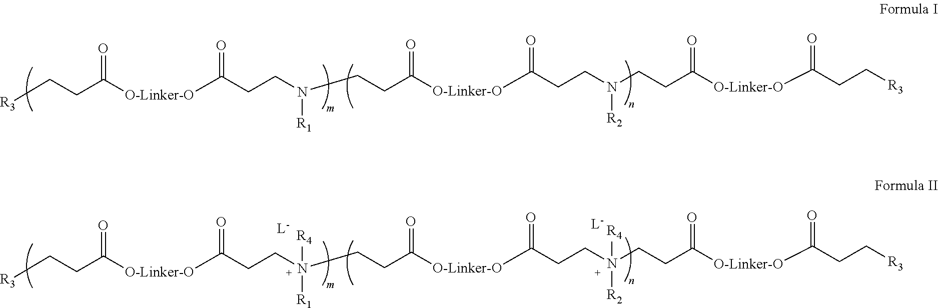
wherein, Linker is selected from one of bisacrylate-containing compounds; R₁ is selected from one of hydrophilic amine compounds; R₂ is selected from one of hydrophobic amine compounds; R₃ is selected from one of linear chain or branched chain of diamino-compounds; R4 is selected from one of halogenated hydrocarbons; L is a monovalent anion; and m:n=1:1˜100.

2. The tissue-selective single-component mRNA delivery carrier according to claim 1, wherein a molecular weight of the poly(β-amino ester) polymer is 2000 Da˜20000 Da.

3. The tissue-selective single-component mRNA delivery carrier according to claim 1, wherein the group of bisacrylate-containing compounds comprise ethylene glycol diacrylate, bis(ethylene glycol) diacrylate, 1,3-butanediol diacrylate, 1,4-butanediol bisacrylate, 1,5-pentanediol diacrylate, 1,6-hexanediol diacrylate, ditriglycol diacrylate, tetraethylene glycol diacrylate, neopentyl glycol diacrylate, 1,10-decanediol diacrylate, 3-methyl-1,5-pentanediol diacrylate, ethoxylated bisphenol A diacrylate, bisphenol A glycerol diacrylate, polyethylene glycol diacrylate, triethylene glycol diacrylate, poly(propylene glycol) diacrylate, fluorescein O,O′-dipropyl diacrylate, and 2-propenoic acid-(2-hydroxy-1,3-propenylidene)bis[oxy(2-hydroxy-3,1-propylidene)] ester.

4. The tissue-selective single-component mRNA delivery carrier according to claim 1, wherein the hydrophilic amine compounds comprise N,N-dimethylethylenediamine, 3-dimethylamino-1-propylamine, 3-diethylaminopropylamine, N,N-diethylethylenediamine, ethanolamine, 4-amino-1-butanol, 2-amino-2-propanol, 2-amino-2methyl-1-propanol, 2-(2-amino) ethoxy) ethanol, N-(3-aminopropyl)morpholine, N-(2-aminoethyl)piperazine, 1-(3-aminopropyl)imidazole, 1-(3aminopropyl)-2-methyl-1-imidazole, 2-amino-2-methyl-1,3-propanediol, 2-amino-1,3-propanediol, 2-amino-5-diethylaminopentane, 1-(2-aminoethyl)piperidine, 3-piperidinylpropanamine, 2-fluoroethylamine, cysteamine, 2-(4-hydroxyphenyl)ethylamine, 4-aminotetrahydrofuran, (3-aminopropyl)trimethoxysilane, 3-aminopropyl(diethoxy)methylsilane, bradypropylamine, 3-pyridinylmethylamine, 5-hydroxydopamine, galactosamine, 1-methoxy-2-propylamine, 2-(ethylthio)ethylamine, 1-pyridinyl-ethylamine, 2-(2-pyridinyl)ethylamine, cysteamine S-phosphate, 6-amino-1-hexanethiol, 2-(2-methylimidazolyl)ethylamine, 2-(1-pyrrol-1-yl)ethylamine, tris(hydroxymethyl)aminomethane, 3-hydroxy-4-methoxybenzene ethanamine, aminoglycol dimethanol, and aminoglycol diethanol.

5. The tissue-selective single-component mRNA delivery carrier according to claim 1, wherein the hydrophobic amine compounds comprise n-hexylamine, n-heptylamine, n-octylamine, n-nonylamine, n-decylamine, n-dodecylamine, n-tridecylamine, n-tetradecylamine, n-hexadecylamine, n-octadecylamine, isoamylamine, iso-octylamine, 6-methyl-1-heptylamine, 2-nonylamine, 2-aminotridecylamine, 6-aminoundecane, 2-ethyl-1-hexylamine, 3-dibutylaminopropylamine, mercapturic undecylamine, oleylamine, and geranylamine.

6. The tissue-selective single-component mRNA delivery carrier according to claim 1, wherein the linear chain or branched chain diamino-compounds comprise ethylenediamine, 1,3-diaminopropane, 1,4-butanediamine, 1,5-pentanediamine, 1,6-hexanediamine, 1,7-diaminoheptane, 1,8-diaminooctane, 1,9-diaminononane, 1,10-diaminodecane, 1,11-undecanediamine, 1,12-dodecanediamine, 1,3-pentanediamine, 1,4-diamino cyclohexane, 1,3-diamino-2-propanol, cystamine, 2,2-bis (aminoethoxy) propane, p-phenylenediamine, 1,2-phenylenediamine, o-phenylenediamine, m-phenylenediamine, m-phenylenediamine, polyethylene glycol bis (amine), 2,2-dimethyl-1,3-propanediamine, 1,2-diaminopropane, 2,2′-oxo-bis(ethylammonium), 1,2-cyclohexanediamine 3,6,9-trioxaundecane-1,11-diamine, 4,9-dioxa-1,12-dodecanediamine, N,N-bis(3-aminopropyl)methanamine, 4,7,10-trioxa-1,13-tridecanediamine, 1,3-cyclohexanedimethanamine, and 4,4′-bisdiphenylphosphine methane (cyclohexylamine).

7. The tissue-selective single-component mRNA delivery carrier according to claim 1, wherein the halogenated hydrocarbons comprise iodomethane, bromomethane, iodoethane, ethyl bromide, bromopropane, iodopropane, 2-iodopropane, 2-iodo-2-methylpropane, 1-iodo-2-methylpropane, isopropyl bromide, bromobutane, 1-bromo-3-methylbutane, 1-bromo-2-ethylbutane, 2-bromo-2-methylpropane, iodobutane, 2-iodobutane, and 1-iodo-3-methylbutane.

8. The tissue-selective single-component mRNA delivery carrier according to claim 1, wherein a method of preparing the poly(β-amino ester) polymer comprises the following procedures:
I, Preparation of the poly(β-amino ester) polymer represented by Formula I
(1) subjecting a bisacrylate Linker, a hydrophilic amine monomer R₁ and a hydrophobic amine monomer R₂ to a Michael addition polymerization reaction in a proportion;
(2) end-capping the polymer prepared in step (1) with a diamine monomer R₃ to obtain the poly(β-amino ester) polymer represented by Formula I;
II, preparation of the poly(β-amino ester) polymer represented by Formula II
(1) subjecting a bisacrylate Linker, a hydrophilic amine monomer R₁ and a hydrophobic amine monomer R₂ to a Michael addition polymerization reaction in a proportion;
(2) end-capping the polymer prepared in step (1) with a diamine monomer R₃ to obtain the poly(β-amino ester) polymer represented by Formula I;
(3) reacting the poly(β-amino ester) polymer represented by Formula I prepared in step (2) with a halogenated hydrocarbon R₄, and converting a tertiary amine in the polymer into a quaternary amine to obtain the poly(β-amino ester) polymer represented by Formula II.

9. The tissue-selective single-component mRNA delivery carrier according to claim 8, wherein in step (1), a molar ratio between the bisacrylate Linker and a total amount of the hydrophilic amine monomer R₁ and the hydrophobic amine monomer R₂ is 1.01˜1.20:1.

10. The tissue-selective single-component mRNA delivery carrier according to claim 8, wherein a reaction solvent for the Michael addition polymerization reaction in step (1) is selected from at least one of N,N-dimethylformamide, dimethyl sulfoxide, tetrahydrofuran, acetonitrile, dioxane, methanol, ethanol, isopropanol, or ethyl acetate; a temperature for the reaction is 25 to 120° C., and a time for the reaction is 2 h to 7 d.

11. The tissue-selective single-component mRNA delivery carrier according to claim 8, wherein in step (2), a molar ratio of the diamine monomer R₃ and the bisacrylate Linker is 0.01˜0.20:1.

12. The tissue-selective single-component mRNA delivery carrier according to claim 8, wherein in step (2), a solvent for the end-capping is selected from at least one of N,N-dimethylformamide, dimethylsulfoxide, tetrahydrofuran, acetonitrile, dioxane, methanol, ethanol, isopropanol, or ethyl acetate; a temperature for the reaction is 25 to 120° C.; and a time for the reaction is 2 h to 24 h.

13. The tissue-selective single-component mRNA delivery carrier according to claim 8, wherein in step (3), an amount of the halogenated hydrocarbon R₄ is 0.1-10 times a total molar amount of the hydrophilic amine monomer R₁ and the hydrophobic amine monomer R₂.

14. The tissue-selective single-component mRNA delivery carrier according to claim 8, wherein in step (3), a reaction solvent is selected from at least one of N,N-dimethylformamide, dimethylsulfoxide, tetrahydrofuran, acetonitrile, dioxane, methanol, ethanol, isopropanol, or ethyl acetate; a temperature for the reaction is 20 to 40° C., and a time for the reaction is 2 h to 72 h.

15. A tissue-selective single-component mRNA delivery system, comprising the delivery carrier according to claim 1, and a mRNA loaded on the delivery carrier.

16. The tissue-selective single-component mRNA delivery system according to claim 15, wherein a method of preparation is as follows: dissolving the poly(β-amino ester) polymer in an organic solvent to obtain an organic phase; diluting the mRNA with an aqueous phase buffer to obtain an aqueous phase of the mRNA; and mixing the organic phase and the aqueous phase of the mRNA to obtain the mRNA delivery system.

17. The tissue-selective single-component mRNA delivery system according to claim 16, wherein the organic solvent is one or more of alcohols, or dimethyl sulfoxide; and a pH of the aqueous phase buffer is 4 to 6.5.

18. The tissue-selective single-component mRNA delivery system according to claim 16, wherein a concentration of the poly(β-amino ester) polymer in the organic phase is 0.1 to 10 mg/mL; and a concentration of the mRNA in the aqueous phase of the mRNA is 5 to 500 ng/μL.

19. The tissue-selective single-component mRNA delivery system according to claim 16, wherein in the mRNA delivery system, a mass ratio of the poly(beta-amino ester) polymer to the mRNA of 1˜200:1.

20. The tissue-selective single-component mRNA delivery system according to claim 15, wherein a method of administrating the mRNA delivery system is selected from one of intravenous, intramuscular, intradermal, subcutaneous, intratumor or intraperitoneal injection.
